(19) United States
(12) Patent Application Publication
(10) Pub. No.: US 2013/0196879 A1
(43) Pub. Date: Aug. 1, 2013

(54) ASSAY SYSTEM

(71) Applicants: Peter Robin Shepherd, Kohimarama (NZ); Rosamund Jane Wealthall, Mount Eden (NZ)

(72) Inventors: Peter Robin Shepherd, Kohimarama (NZ); Rosamund Jane Wealthall, Mount Eden (NZ)

(21) Appl. No.: 13/640,022

(22) PCT Filed: Apr. 7, 2011
(86) PCT No.: PCT/NZ2011/000050

§ 371 Date: Feb. 27, 2013

(30) Foreign Application Priority Data
Apr. 8, 2010 (NZ) 584497

(51) Int. Cl. G01N 33/50; C12Q 1/48

(52) U.S. Cl. CPC G01N 33/5008; C12Q 1/485
USPC 506/15; 506/23

(57) ABSTRACT

The invention provides a method of forming a plurality of re-constitutable doses of at least one drug in a plurality of wells, the method including the steps of (i) placing a known amount of said drug in a suitable carrier to form a first composition having a known concentration (ii) placing at least two selected amounts of that first composition into individual wells and (iii) converting the first composition into a transportable form that can later be converted into a second composition having a known concentration and (iv) sealing the wells.

### TECHNICAL FIELD

The invention relates to a method of providing an array of drugs that target cell signalling molecules. In particular the array provides serial dilutions of such drugs that span the concentration range including the EC₅₀ and IC₅₀ appropriate for the molecular target of the drugs. The invention also relates to an array of drugs that target cell signalling molecules, the array serial dilutions of such drugs that span the concentration range including the EC₅₀ and IC₅₀ appropriate for the molecular target of the drugs. In particular, the invention also relates to the provision of drugs in a transportable and re-constitutable form, the drugs being useful to provide an array having said serial dilutions or to provide a plurality of dilutions to set concentrations.

### BACKGROUND ART

Drugs having an inhibition effect are a powerful tool to investigate cell signalling pathways, but must be used carefully to produce results of value. In particular, an inhibitor must be used at a concentration that selectively blocks one pathway but not others. This concentration at which a drug selectively blocks 50% of the target signalling pathway or cellular processes in the cell is referred to as “Effective Concentration₅₀”, or “EC₅₀”. The concentration at which a drug selectively blocks 50% of the target in in-vitro assays is referred to as “Inhibitory Concentration₅₀”, or “IC₅₀”. There are many inhibitors available on the market, but it can be difficult to obtain reliable estimates of their EC₅₀s for various applications. For individual researchers, it is frequently a time-consuming task to develop their own EC₅₀s and given the small amounts of drug used to determine EC₅₀, the cost of such tasks can be prohibitive.

It is also an issue to be able to transport drugs in a form that can be accurately reconstituted on-site by a user. This can be important when looking to provide individual researchers arrays of inhibitors that can be used to cover EC₅₀s for various applications (as referred to above) and/or when looking to provide accurate dilutions of drugs for users in the field. For example, users in the field could include mobile hospitals (military or otherwise) or disaster relief where a plurality of doses of pharmaceuticals may be needed that can be transported readily and reformed accurately.

### OBJECT OF THE INVENTION

It is an object of the invention to provide a method of providing an array of drugs that specifically target cell signalling molecules and/or to provide an array of drugs that specifically target cell signalling molecules or to provide the means and a method to provide drugs in a transportable and reconstitutable form and/or to provide the public with a useful choice.

### SUMMARY OF THE INVENTION

In a first aspect the invention provides a method of forming a plurality of re-constitutable doses of at least one drug in a plurality of wells, the method including the steps of (i) placing a known amount of said drug in a suitable carrier to form a first composition having a known concentration (ii) placing at least two selected amounts of that first composition into individual wells and (iii) converting the first composition into a transportable form that can later be converted into a second composition having a known concentration and (iv) sealing the wells.

Preferably, the plurality of re-constitutable doses of at least one drug is an array of a plurality of drugs that target cell signalling molecules, and steps (i) and (ii) include determining a series of dilutions for each of the selected drugs that span the EC₅₀ of the molecular target of the selected drugs and dispensing an amount of each of the selected drugs into a series of wells such that when a fixed amount of the selected drugs is transferred from each well to a series of fixed volumes of the molecular target, the final range of concentrations created spans the EC₅₀ of the molecular target (determined previously), and step (iii) includes purging the series of wells with a suitable gas prior to sealing the wells.

Preferably, the plurality of re-constitutable doses of at least one drug is an array of at least one drug and step (i) includes determining the desired concentration(s) of the selected drug(s) and forming a first composition of the selected drug(s) having that desired concentration.

Preferably, the first composition is converted into a transportable form by freezing the first composition and the first composition and the second composition are the same.

Preferably, the first composition is converted into a transportable form by evaporating the suitable carrier from the first composition such that the drugs are converted into a powder or crystalline form.

Preferably, the first composition is converted into a transportable form by centrifuging and evaporating the suitable carrier from the first composition such that the drugs remain in their individual wells.

Preferably, the first composition is freeze dried.

Preferably the carrier is removed by centrifuging under vacuum.

Preferably, the wells are sealed first and then the amount(s) of the first composition in the wells are converted into a transportable form that can later be converted into a second composition having a known concentration by freezing.

Preferably, the amount(s) of the first composition in the wells are converted into a transportable form that can later be converted into a second composition having a known concentration by evaporation and the wells are then sealed.

Preferably the transportable form is a powder or crystalline form of the drug.

Preferably the selected drug is selected from kinase inhibitors, more preferably PI3 kinase and MAP kinase inhibitors.

Preferably the selected drug is selected from antibiotics, pain relief drugs, anti-inflammatory drugs, trauma medication, and psychiatric drugs.

Preferably the suitable solvent is selected from ethanol, methanol, water, DMF or DMSO.

Preferably the first and the second compositions are solutions, suspensions, dispersions or emulsions.

Preferably the wells are purged with an inert gas, more preferably nitrogen, argon or the like, prior to sealing.

Preferably the seals allow opening and re-sealing of individual or a plurality of wells.

Preferably the seals reseal the wells following puncture access (e.g. by syringe needle).

Preferably the seals are resistant to the solvent used.

Preferably the seals are capable of at least minimising evaporation and cross-contamination.

Preferably the array is formed of a polypropylene or polystyrene material.

Preferably the array is provided in a plate form or as a series of well strips.

Preferably the array is a combination of tubes and a frame.

Preferably the array includes 96 wells.

Preferably the array is placed in a suitable temperature controlled container for transport.

Preferably step (iii) includes evaporating the suitable carrier from the first composition such that the drugs are converted into a powder form and remain in their individual wells, and are later converted into the second composition using the same carrier as used in the first composition.

Preferably step (iii) includes evaporating the suitable carrier from the first composition such that the drugs are converted into a powder form and remain in their individual wells, and are later converted into the second composition using a different carrier to that used in the first composition.

In another aspect the invention provides a method of providing an array of drugs that target cell signalling molecules, the method including the steps of:
A) selecting a plurality of drugs each of which targets a cell signalling molecule;
B) determining a series of dilutions for each of the selected drugs that span the EC₅₀ of the molecular target of the selected drugs;
C) dispensing an amount of each of the selected drugs into a series of wells;
D) transforming the amount of each of the selected drugs in the wells into a transportable form capable of reconstitution;
E) purging the wells with a suitable gas;
F) sealing the wells; and
   wherein, the amount of each of the selected drugs dispensed in the wells is such that, when the selected drugs are reconstituted, and a fixed amount of the selected drugs is transferred from each well to a series of fixed volumes of the molecular target, the final range of concentrations created spans the EC₅₀ of the molecular target (determined in B).

Preferably the final range of concentrations created also spans the IC₅₀ of the molecular target of the selected drugs.

Preferably the molecular target is an appropriate cell or tissue culture media.

Preferably the drug is dispensed into the well in a solution of suitable diluent, most preferably DMSO, DMF, water, methanol, or ethanol.

Preferably in step D the diluent is removed by evaporation to leave a powder such that when a fixed amount of diluent is later added to the wells, a series of concentrations of the drug is created for transfer to the fixed volumes of the molecular target.

Preferably the solvent is removed by freeze drying or by evaporation.

Preferably solvent evaporation is vacuum assisted.

Preferably solvent evaporation is centrifuge assisted.

Preferably solvent evaporation is by centrifuging under vacuum.

Preferably the transportable form of the drug is a solid form.

Preferably the transportable form of the drug is a powder or crystalline form.

Preferably the method includes reconstitution by appropriate re-suspension of the selected drug in the well and subsequent dilution of the selected drug in the cell/tissue culture media.

Preferably the method includes reconstitution by appropriate dissolution of the selected drug in the well and subsequent dilution of the selected drug in the cell/tissue culture media.

Preferably the series of wells is contained in a multiwall plate containing a plurality of series of different drugs thus allowing the creation of an array that covers the EC50, and optionally the IC50, of a plurality of different cell signalling molecules.

Preferably the selected drug is selected from kinase inhibitors, more preferably PI3 kinase and MAP kinase inhibitors.

Preferably the drug and solvent are dispensed in separate aliquots into the well.

Preferably the wells are purged with an inert gas, more preferably nitrogen, argon or the like.

Preferably the seals allow opening and re-sealing of individual or a plurality of wells.

Preferably the seals reseal the wells following puncture access (e.g. by syringe needle).

Preferably the seals are resistant to the solvent used.

Preferably the seals are capable of at least minimising evaporation and cross-contamination.

Preferably the dilutions span 1/10th of the selected drug's IC₅₀ to 100× that drug's IC₅₀.

Preferably a selected drug will be sequentially diluted to provide a well strip including a sequence of at least 5 wells, more preferably 7 wells, the array being formed from a series of well strips.

Preferably the well strips also include wells including the diluent as a negative control for each of the sequential drug dilutions.

Preferably the array includes a series of 8 well strips, the well strips including a sequence of drug dilutions and a negative control.

Preferably the array includes 96 wells.

Preferably the selected drugs and/or the diluent are dispensed to the wells automatically (e.g. robotically) or manually.

Preferably dispensing occurs in a manner to minimise freeze-thaw cycles.

Preferably each well, optionally including the well containing the negative control, will include sufficient solution for up to at least 3 assay points, more preferably up to at least 5 assay points.

Preferably the array is formed of a polypropylene or polystyrene material.

Preferably the array is provided in a plate form.

Preferably the array is a combination of tubes and a frame.

Preferably the array is used to determine metabolic pathway activation.

Preferably the array is used to determine cell signalling pathway activation.

Preferably cell signalling pathway activation is determined via methods that detect the posttranslational modification of proteins by phosphorylation.

Preferably cell signalling pathway activation is determined via methods that detect the activity of enzymes

Preferably cell signalling pathway activation is determined via methods that detect the changes is metabolism in response to hormones or cytokines.

Preferably the array, including the sequentially diluted drugs in sealed wells, is placed in a suitable temperature controlled container, for transport.

In another aspect the invention provides a method of providing an array of drugs that target cell signalling molecules, the method including the steps of:
A) selecting a plurality of drugs each of which targets a cell signalling molecule;
B) determining a series of dilutions for each of the selected drugs that span the EC₅₀ of the molecular target of the selected drugs;
C) dispensing an amount of each of the selected drugs into a series of wells;
D) purging the wells with a suitable gas;
E) sealing the wells; and
   wherein, the amount of each of the selected drugs dispensed in the wells is such that when a fixed amount of the selected drugs is transferred from each well to a series of fixed volumes of the molecular target, the final range of concentrations created spans the EC₅₀ of the molecular target (determined in B).

Preferably the final range of concentrations created also spans the IC₅₀ of the molecular target of the selected drugs.

Preferably the molecular target is an appropriate cell or tissue culture media.

Preferably the drug is dispensed into the well in a solution of suitable diluent, most preferably DMSO, DMF, water, methanol, or ethanol.

Preferably the diluent is removed by evaporation or freeze drying to leave a powder such that when a fixed amount of diluent is later added to the wells, a series of concentrations of the drug is created for transfer to the fixed volumes of the molecular target.

Preferably diluent evaporation is vacuum assisted.

Preferably diluent evaporation is centrifuge assisted.

Preferably diluent evaporation is by centrifuging under vacuum.

Preferably the transportable form of the drug is a powder or crystalline form.

Preferably the method includes appropriate re-suspension of the selected drug in the well and subsequent dilution of the selected drug in the cell/tissue culture media.

Preferably the method includes appropriate dissolution of the selected drug in the well and subsequent dilution of the selected drug in the cell/tissue culture media.

Preferably the series of wells is contained in a multiwall plate containing a plurality of series of different drugs thus allowing the creation of an array that covers the EC50, and optionally the IC50, of a plurality of different cell signalling molecules.

Preferably the selected drug is selected from kinase inhibitors, more preferably PI3 kinase and MAP kinase inhibitors.

Preferably the drug and solvent are dispensed in separate aliquots into the well.

Preferably the wells are purged with an inert gas, more preferably nitrogen, argon or the like.

Preferably the seals allow opening and re-sealing of individual or a plurality of wells.

Preferably the seals reseal the wells following puncture access (e.g. by syringe needle).

Preferably the seals are resistant to the solvent used.

Preferably the seals are capable of at least minimising evaporation and cross-contamination.

Preferably the dilutions span 1/10th of the selected drug's IC₅₀ to 100× that drug's IC₅₀.

Preferably a selected drug will be sequentially diluted to provide a well strip including a sequence of at least 5 wells, more preferably 7 wells, the array being formed from a series of well strips.

Preferably the well strips also include wells including the diluent as a negative control for each of the sequential drug dilutions.

Preferably the array includes a series of 8 well strips, the well strips including a sequence of drug dilutions and a negative control.

Preferably the array includes 96 wells.

Preferably the selected drugs and/or the diluent are dispensed to the wells automatically (e.g. robotically) or manually.

Preferably dispensing occurs in a manner to minimise freeze-thaw cycles.

Preferably each well, optionally including the well containing the negative control, will include sufficient solution for up to at least 3 assay points, more preferably up to at least 5 assay points.

Preferably the array is formed of a polypropylene or polystyrene material.

Preferably the array is provided in a plate form.

Preferably the array is a combination of tubes and a frame.

Preferably the array is used to determine metabolic pathway activation.

Preferably the array is used to determine cell signalling pathway activation.

Preferably cell signalling pathway activation is determined via methods that detect the posttranslational modification of proteins by phosphorylation.

Preferably cell signalling pathway activation is determined via methods that detect the activity of enzymes

Preferably cell signalling pathway activation is determined via methods that detect the changes is metabolism in response to hormones or cytokines.

Preferably the array, including the sequentially diluted drugs in sealed wells, is placed in a suitable temperature controlled container, for transport.

In another aspect the invention provides an array including a plurality of well strips, each well strip containing a selected drug which targets a cell signalling molecule, each well strip including a plurality of wells containing a either a sequence of dilutions of the selected drug that span the EC₅₀, and optionally the IC₅₀, of the molecular target of the drug, or the selected drug in a form capable of dilution to create a sequence of dilutions of the selected drug that span the EC₅₀, and optionally the IC₅₀, of the molecular target of the drug, each well also including an inert gaseous environment and being sealed to contain the selected drugs in that inert environment.

Preferably the inert gaseous environment is nitrogen, argon or like inert gas.

Preferably the solvent for the drugs is DMSO, DMF, water, methanol, or ethanol.

Preferably the form of the selected drug capable of dilution to create a sequence of dilutions of the selected drug that span the EC₅₀ and preferably the IC₅₀ of the molecular target of the drug is a powder or crystalline form.

Preferably the form of the selected drug capable of dilution to create a sequence of dilutions of the selected drug that span the EC₅₀ and preferably the IC₅₀ of the molecular target of the drug, is created by evaporation.

Preferably the evaporation is vacuum assisted, more preferably centrifuging under vacuum.

Preferably the seals are resistant to the diluent used.

Preferably the seals allow opening and re-sealing of individual or a plurality of wells.

Preferably the seals are capable of resealing the wells following puncture access (e.g. by syringe needle) to the diluted drugs therein.

Preferably the series of dilutions span 1/10th of the drug's IC₅₀ and EC₅₀ to 100× that drug's IC₅₀ and EC₅₀

Preferably the array also includes wells including the diluent solvent as a negative control for each of the sequential drug dilutions.

Preferably each well, optionally including the well containing the negative control, will include sufficient solution for up to at least 3 assay points, more preferably up to at least 5 assay points.

Preferably the array is formed of a polypropylene or polystyrene material.

Preferably the array is provided in a plate form.

Preferably the array is a combination of tubes and a frame.

Preferably the array includes 96 wells.

In a third aspect the invention provides a kit of parts, the kit including an array according to any one of the previous aspects of the invention together with a suitable temperature controlled and/or mechanical shock controlled container allowing transport of the array.

### FIGURES

FIG. 1 shows in diagrammatic form an example of an array (PI3K) according to the invention.

FIG. 2 shows, again in diagrammatic form, an example of a method of providing an array (PI3K) and using that array to assess pathway activation.

FIG. 3 shows, again in diagrammatic form, an example of using an array (PI3K) to assess pathway activation.

FIGS. 4 and 5 show a comparison of effectiveness of the inhibitor drug CI1040 evaporated from ethanol solution and reconstituted in DMSO (FIG. 4) and directly dissolved in DMSO (FIG. 5).

FIGS. 6 and 7 show a comparison of effectiveness of the inhibitor drug PD325901 evaporated from ethanol solution and reconstituted in DMSO (FIG. 6) and directly dissolved in DMSO (FIG. 7).

FIGS. 8 and 9 show a comparison of effectiveness of the inhibitor drug PD98059 evaporated from ethanol solution and reconstituted in DMSO (FIG. 8) and directly dissolved in DMSO (FIG. 9).

### DETAILED DESCRIPTION

The arrays according to the present invention allow the transport of a plurality of drugs for a number of uses in an efficient manner. They can feature highly potent drugs designed to block a range of metabolic pathways. In particular, the arrays use highly potent drugs that inhibit a range of key signalling metabolic pathways. These arrays are extremely easy to use, can include both well-characterised and novel inhibitors, and offer the opportunity to sample many different inhibitors without buying each inhibitor separately. The arrays therefore provide a quick straightforward and affordable way to simultaneously explore several signalling pathways.

The arrays can include a variety of both well-characterised and novel inhibitor drugs. The inhibitors can be isoform-specific, allowing fine dissection of activated signalling pathways. Combinations of these isoform-specific inhibitors can be provided in the array. These pre-made combinations make it possible to completely inhibit a target with ease. Other inhibitors can then block associated pathways, enabling new connections between signalling pathways to be investigated.

The pre-made combination array can be provided to the user for use as desired. The ability to package the array and to transport it to the user allows flexibility of use and allows the user to conduct research in locations of choice. Accuracy of the array when used in the location of choice is important therefore the ability to reconstitute the drugs accurately in the array at that location is also important. The combination can be a standard array or can be produced to target particular combinations of pathways. The array can be used, for example, to determine the activation status of cell signalling pathways using western blot analysis or ELISA assays. Preferably the array is used to determine cell signalling pathway activation, preferably via methods that detect the posttranslational modification of proteins by phosphorylation, methods that detect the activity of enzymes, and/or methods that detect the changes is metabolism in response to hormones or cytokines.

The pre-made array can also be used to transport drugs in a form that can be accurately reconstituted on-site by a user. This would allow field hospitals, for example, to be provided with accurately reconstitutable pharmaceuticals (such as pain relief (e.g. morphine), antibiotics etc) of known concentration(s). Pharmaceuticals that field hospitals might use for infectious diseases such as cholera, typhoid, amoebic dysentery include doxycycline, cyprofloxacin, azithromycin, ampicillin, mitronidazole. For trauma, the coagulants wilate and Atryn. Anti-inflammatory medications include corticosteriods and non-steroidal anti-inflammatories (aspirin, ibuprofen).

As will be readily apparent to the skilled person these methods are by way of example only and a variety of other option could be used as desired.

The invention, in a general sense, therefore provides a method of forming a plurality of individual re-constitutable doses of at least one drug in a plurality of wells. The plurality of wells can be usefully referred to as an array. The method includes the steps of (i) placing a known amount of said drug in a suitable carrier to form a first composition having a known concentration (ii) placing at least two selected amounts of that first composition into individual wells and (iii) converting the first composition into a transportable form that can later be converted into a second composition having a known concentration and (iv) sealing the wells.

As will be apparent, depending on the method by which step (iii) occurs, step (iii) and step (iv) can happen in either order. Conversion can occur before or after the wells are sealed, depending on the manner of conversion (e.g. if the first composition is frozen then either order is an option, if the first composition is converted into a powder form then this would happen before the wells are sealed).

In a preferred form, the plurality of re-constitutable doses of at least one drug is an array of a plurality of drugs that target cell signalling molecules, and steps (i) and (ii) include determining a series of dilutions for each of the selected drugs that span the EC₅₀ of the molecular target of the selected drugs and dispensing an amount of each of the selected drugs into a series of wells such that when a fixed amount of the selected drugs is transferred from each well to a series of fixed volumes of the molecular target, the final range of concentrations created spans the EC₅₀ of the molecular target (determined previously), and step (iii) includes purging the series of wells with a suitable gas prior to sealing the wells.

In one preferred form therefore the invention provides a method of providing an array of drugs that target cell signalling molecules, the method including the steps of:
A) selecting a plurality of drugs each of which targets a cell signalling molecule;
B) determining a series of dilutions for each of the selected drugs that span the EC₅₀ of the molecular target of the selected drugs;
C) dispensing an amount of each of the selected drugs into a series of wells;
D) transforming the amount of each of the selected drugs in the wells into a transportable form capable of reconstitution;
E) purging the wells with a suitable gas;
F) sealing the wells; and
   wherein, the amount of each of the selected drugs dispensed in the wells is such that, when the selected drugs are reconstituted, and a fixed amount of the selected drugs is transferred from each well to a series of fixed volumes of the molecular target, the final range of concentrations created spans the EC₅₀ of the molecular target (determined in B).

In another preferred form, the plurality of drug doses is an array of at least one selected drug and step (i) includes determining the desired concentration(s) of the selected drug(s) and forming a first composition of the selected drug(s) having that desired concentration. This option allows for the array to be able to transport selected drugs in a form that can be accurately reconstituted on-site by a user. This can be important when looking to provide accurate dilutions of drugs for users in the field. For example, users in the field could include mobile hospitals (military or otherwise) or disaster relief where a plurality of doses of pharmaceuticals may be needed that can be transported readily and reformed accurately. In such a case the same amount of the same selected drug may be in the wells of the array so that a user knows that reconstitution of the drug (i.e. into the second composition) using a set amount of carrier will provide a desired concentration of the drug for use (for injection, infusion, or oral application for example). Alternative options such as different amounts of drug or different drugs could also be used if desired.

Reference to “transportable form” means that the first composition (i.e. the first dilution of the drug with a suitable carrier) has been changed into a form that is more conducive to transportation than the initial liquid form. While a liquid form does have some advantages, in that the end user does not have to reconstitute the composition, this option has transportation issues, such as weight, and also the potential for the carrier/diluent to have a deterioration effect on the seal used on the well. Transport of liquids is also more difficult as security of the sample (e.g. leakage) is more difficult than when transporting a solid material. Useful options would therefore be advantageous.

The amount of drug provided in each well is related to the potency of that compound (i.e. the drug) when it is used. The more potent the compound, the lower the concentration of the solution, and the smaller the amount of powder in the well. For a potent compound, such as CI1040, the first composition is at a concentration of 1 mM, and to provide a final volume of 35 μl (at least 5 assay points), the total amount of powder is 0.017 mg. This amount of CI1040 is too small to see, and therefore cannot be measured accurately using a standard balance. Further, the lowest concentration of CI1040 provided on the array is 1 μM, or one-thousandth of this total amount (0.000017 mg). At these levels it is very difficult to accurately provide a measured amount of drug for dilution/reconstitution at a later place and time. Further, a drug in powder form that is simply placed in a well for dilution at a later time can also result in inaccuracies as the powder placed in the well will not readily be retained deep enough in the well and thus a portion of the drug may not be present in the dilution. The powder may adhere to the underside of the seal used or may simply be resting too high up the sides of the walls of the well. By dissolving/suspending it in a solution/suspension then evaporating the carrier to provide a transportable form of the drug, the inventors have found that a suitably accurate measured amount of a drug can be provided and the powder (or crystalline) solid thus formed is positioned and retained deep enough into the well to be captured later on dilution. A vacuum is preferably used to assist evaporation as this will speed up the conversion step to the transportable form, thus assisting the efficiency of the overall process. A quick evaporation will usually result in a powder form of the drug being present in the well, and a slower evaporation can result in crystals of the drug in the well.

The inventors have also found that coupling the evaporation step with centrifuging significantly increases the security of the drug in the well by forcing the drug as far as possible into the base of the well thus reducing the potential for the drug in the well to be missed on dilution later (i.e. reconstitution). The most preferred option is to conduct the evaporation step by centrifuging under a vacuum.

The inventors have found that the use of the evaporative technique according to the invention allows accurate and effective results to be achieved on reconstitution of the drug in a desired solvent that are comparable to when the drug is directly formulated in the desired solvent. The result is that this aspect of the invention provides particular advantages to the public that were not previously available

The particularly preferred option is therefore for the diluent used to dissolve or suspend the drug is a carrier that is capable of being evaporated such that the drugs are converted into a powder form and remain in their individual wells. Evaporation of the carrier has the particular benefit that the drug being is left in a dry and relatively light form in the well. Thus weight concerns are avoided as are some of the more complicated environment issues (although temperature and light issues may need to be addressed to mitigate deterioration over time). Options to evaporate the carrier include freeze drying and the use of carriers that are volatile enough to evaporate (preferably ethanol, methanol, or water, although other suitable volatile carriers as would be known to the skilled person could be used). A vacuum is preferably used to assist evaporation as this will speed up the conversion step, thus assisting the efficiency of the overall process. A quick evaporation will usually result in a powder form of the drug being present in the well, and a slower evaporation can result in crystals of the drug in the well. It is also most preferred to include centrifuging when evaporating the suitable carrier from the first composition so that the drugs remain in their individual wells. Centrifuging with evaporation means that the drugs in powder (or other) form are forced to the base of the well and away from the wall and seal of the well (once the well is sealed). This is of particular advantage as it reduces the likelihood that, when transporting the array including the drug in a dry form, the drug, or some of the drug, will move onto the walls of the well. This can have the effect that when reforming the drug into the second composition, some of the powder is missed therefore accuracy is detrimentally affected.

That loss of accuracy, depending on the final use of the drug, may not always be of concern, but in many cases it will be critical. Preferably, therefore, in order to meet such problems, the first composition will be converted into the transportable form by centrifuging in a vacuum. The inventors have found that the use of the evaporative technique according to the invention allows accurate and effective results to be achieved that are comparable to when the drug is directly formulated in the desired solvent. The result is that this aspect of the invention provides particular advantages to the public that were not previously available.

A less preferred alternative is freezing the first composition to convert that composition from a liquid to a solid (i.e. ice). Carriers such as DMSO or DMF would be options. In this case the first composition and the second composition, once it is formed by the ultimate user, will be the same. This option is not as preferred as evaporation as, while this option of solidifying the liquid has the advantage of simplicity, there can still be issues with transporting a frozen array from a weight perspective as well as the need to keep the well contents frozen. The frozen form is, however, less likely to impact on the seal integrity and will have less sample security issues and therefore may be an option of use in some circumstances.

A number of centrifuge options are available as would be known to the skilled person. These include, by way of example, Savant SpeedVac Concentrator #SPD111V, and MiVac Genevac #23050HOO. Such centrifuges are operated at the fixed speed and level of vacuum of the units. The temperature is adjustable on some options (e.g. Savant SpeedVac Concentrator #SPD111V), and it is preferable during manufacture of the array, to set this the lowest suitable setting (of 35° C. in the Savant SpeedVac Concentrator #SPD111V) to minimize heating of the compound. The centrifuge speed and level of vacuum used is therefore standard and would be within the ability of a person skilled in the art to determine.

There are a variety of suitable diluent carriers that could be used in the first and second compositions, dependant on the conversion step chosen. Suitable carriers will preferably be solvents but can also form suspensions, dispersions, emulsions or like compositions with the drug. Suitable carriers can be selected from ethanol, methanol, water, DMF or DMSO for example. For the evaporation option (preferably centrifuge under vacuum) ethanol, methanol and water are preferred. For reconstitution, the carrier can be as desired by the user (preferably DMSO/DMF) and need not be the same carrier as initially used. The main issues with the composition, particularly the composition formed by reconstitution of the drug, is dispersion of the drug through the composition so that acceptable accuracy is achieved. For this reason, the formation of solutions is usually preferred.

There are a variety of drugs that could be used in the array. The inhibitor drugs selected that target cell signalling molecules will be selected from kinase inhibitors, and more preferably PI3 and MAP kinase inhibitors. The drug can also be selected from any suitable pharmaceutical that will not be impaired by the process. Such drugs can include antibiotics, pain relief drugs (e.g. morphine), anti-inflammatory, trauma medication, psychiatric drugs or the like.

The invention, in an alternative form, also provides a method of providing an array of drugs that target cell signalling molecules, the method including the steps of:
A) selecting a plurality of drugs each of which targets a cell signalling molecule;
B) determining a series of dilutions for each of the selected drugs that span the EC₅₀ of the molecular target of the selected drugs;
C) dispensing an amount of each of the selected drugs into a series of wells;
D) purging the wells with a suitable gas;
E) sealing the wells; and
   wherein, the amount of each of the selected drugs dispensed in the wells is such that when a fixed amount of the selected drugs is transferred from each well to a series of fixed volumes of the molecular target, the final range of concentrations created spans at least the EC₅₀ of the molecular target (determined in B).

It is preferred that the series of dilutions for each of the selected drugs also spans the IC₅₀ of the molecular target of the selected drugs. It is also preferred that the molecular target is in an appropriate cell or tissue culture media.

The inhibitor drugs, used in the array to create the final range of concentrations that spans at least the EC₅₀ of the molecular target, are preferably provided in a DMSO solution, which is later diluted in cell culture medium and added to cells, or diluted in buffer.

This method can be adapted to provide the array with the drugs converted into a more transportable form, for example where the inhibitor drugs are diluted in ethanol (or another solvent/carrier that is capable of evaporation) as discussed earlier. An amount of each is dispensed into the wells and the ethanol is then evaporated off leaving a known amount of the inhibitor drug in the wells to convert the amount dispensed into a transportable form (as referred to above). Evaporation is preferably done by centrifuge under vacuum or freeze drying as discussed previously. When a fixed amount of solvent (e.g. DMSO) is later added to the powder in the wells, a series of concentrations of the drug is created for transfer to the fixed volumes of the molecular target.

Whether adapted to provide the array with the drugs converted into a more transportable form or not, each inhibitor drug will preferably be provided in well strips (the strips including a plurality of wells) in the array in a range of concentrations, so that the optimal concentration can be found without effort. Alternatively, the series of wells is contained in a multiwall plate containing a plurality of series of different drugs thus allowing the creation of an array that covers the EC₅₀ of a plurality of different cell signalling molecules.

The range of concentrations for a particular inhibitor drug provided in the array preferably spans from one-tenth of the IC₅₀, through five-fold or ten-fold dilutions, up to one hundred times the IC₅₀. This large range ensures that the targeted pathway is specifically blocked. No molarity calculations are required by the user, which facilitates error-free experimentation. This is important when targeting cell signalling molecules because if used at incorrect concentrations, misleading results can be generated. For example, many inhibitor drugs when used at very high concentrations will block off-target pathways. Accuracy of reconstitution in this case is of importance to the user.

The selected drug and diluent used in the various embodiments of the invention are preferably dispensed in separate aliquots into the series of wells, although it is of course possible for the dilutions to be completed prior to dispensing the solution into the wells. The sequence of dilutions for a particular drug will form a well strip for that drug and the array will have a series of these strips. Dispensing of the aliquots into the wells can be completed automatically (e.g. robotically or the like) or manually.

The selected drugs may be sequentially diluted to provide a well strip having a suitable number of wells for an adequate range of concentrations to be covered, when sequential dilutions are needed (e.g. target cell signalling molecules). The well strip could of course simply form part of a multiwall plate that includes a number of well strips. Usually the strip will be a sequence of at least 5 wells, more preferably 7 wells, in each well strip.

The array of drugs when used to target cell signalling molecules should preferably also include wells including the diluent as a negative control for each of the sequential drug dilutions, although this is optional. Preferably, therefore, the well strips include a sequence of 7 drug dilutions and a negative control. The array in a preferred form will include 96 wells, formed from 12 well strips each having 8 wells. Again, this is not intended to be limiting as the number of wells in a well strip could vary as could the number of well strips in an array.

The amount of solution (drug+diluent) in each well should be sufficient, on transfer to molecular target, to provide adequate assay points to the user. Preferably this will be up to at least 3 assay points, more preferably up to at least 5 assay points. The molecular target is preferably in an appropriate cell or tissue culture media as would be known to the skilled person. This culture media would contain fixed volumes for accurate assessment of the assay points. Of course, when the array is used to transport pharmaceuticals (e.g. antibiotics etc) the amount of drug in the wells should be sufficient to be reformed using a suitable carrier (e.g. water, saline solution) into a pharmaceutical composition (drug+diluent) suitable for purpose.

The working volume of the wells in a standard array plate is about 200 μl (although this could vary, for example a Deepwell plate holds 2 mls—so may be more suitable for drug transfer). The limitations are the amount of drug required per use, and the solubility of the drug in the carrier (e.g. ethanol). For example, if the amount of the drug required in the well is 1 mg, and that amount of drug needs 20 mls ethanol to dissolve, then the drug would be dissolved in the 20 mls of ethanol, 2 mls of the ethanol/drug solution would be put in the well, evaporated, then repeated 9 more times (to get all the drug in the well).

In order for the drug in the wells to be able to be maintained at the desired concentration/amount for a suitable period to allow for storage and transportation while mitigating deterioration, the wells should be purged with suitable gas, such as an inert gas (e.g. nitrogen, argon or like gas). The wells are then sealed to contain the desired gaseous environment in the well, minimise evaporation, and preferably also to reduce (or preferably eliminate) the risk of cross-contamination between the wells in the array. One option for purging the wells is that the plates, containing the wells, are placed inside a sealable, preferably plastic, box which is flushed with gas via a hose from the gas tank containing the inert gas, then the box is sealed for a period of time, to allow equilibration.

The seals should also preferably be resistant to any diluent used to maintain the integrity of the seal. In addition, it is preferred that the well and the seals block light from impacting on the drug, as this can, in some circumstances, also cause drug deterioration.

The seals should, of course, allow access to the diluted drugs in the sealed wells. Preferably, therefore, the seals will allow opening (for reconstitution) and re-sealing of individual or a plurality of wells. Alternative options are of course available such as the seals being of a type to allow resealing following puncture access (e.g. by syringe needle).

In another aspect the invention provides an array including a plurality of well strips, each well strip containing a selected drug which targets a cell signalling molecule, each well strip including a plurality of wells containing a either a sequence of dilutions of the selected drug that span the EC₅₀, and optionally the IC₅₀, of the molecular target of the drug, or the selected drug in a form capable of dilution to create a sequence of dilutions of the selected drug that span the EC₅₀, and optionally the IC₅₀, of the molecular target of the drug, each well also including an inert gaseous environment and being sealed to contain the selected drugs in that inert environment.

The array, in a preferred form will be formed of polypropylene, polystyrene, or like, material and will be a unitary structure. Preferably the array will be in a plate form.

Alternatively, the array could be a combination of tubes (e.g. test tubes or the like) and a frame to hold those tubes. The tubes would perform the function of the wells, and the frame would hold those tubes in an array structure.

The pre-made array will, in a preferred form, be placed in a suitable temperature controlled container, for transport to the user. This could contain frozen material such as ice, although if the final array being transported includes the drug(s) frozen in DMSO then it would be best to keep it at 4 degrees as this would ensure the DMSO remains solid. The container should also be able to resist mechanical shock. Issues such as this would be well known to the skilled person.

A pre-made array could also be used to transport a single drug in a form that can be accurately reconstituted on-site by a user. The array could take the form of a range of doses of a single drug, the range being appropriate for different patients such as infants, children, adults, the elderly, pregnant women, those on other medication, etc. To maintain sterility the array should be fully sealed, and the drugs reconstituted solely by puncture access (e.g. by syringe needle). The appropriate solvent for the drugs (sterile water, physiologic saline) could be injected, and then once the drug was fully dissolved, used to withdraw the reconstituted drug. The drug could then be directly administered (e.g. intravenously, intramuscularly, subcutaneously) or further dissolved in an appropriate solvent (saline, glucose in water) in an IV bag or similar. The individual units of the array would be single-use only. Syringes and vials of sterile water or other solvent could be provided as part of the array, for example set amount of the appropriate solvent could also be provided in array wells, accessible in a similar sterile manner to the drug wells.

Such an array could take the form of combinations of drugs suitable for different infectious diseases. For example, cholera treatment could include cyprofloxacin plus doxycycline in the same unit of the array, while typhoid treatment could include cyprofloxacin plus ampicillin. Such combinations would be well known to a skilled person in this field.

The ability to provide accurate amounts of the desired drug in a transportable form via the evaporative technique of the present invention allows such options to be available to the public.

The present invention therefore also provides a kit of parts, the kit including an array according to the present invention together with a suitable, preferably temperature controlled, container, allowing transport of the array. Preferably the container would alternatively be, or would also be, resistant to mechanical shock.

The following is a description, by way of general example, of preparing an array according to an embodiment of the present invention. This preparation process uses a process of evaporation to prepare the drugs, in this case inhibitor drugs that target cell signalling molecules, for transport.

### Method of Production:

Inhibitor compounds in powder form are dissolved in pure ethanol to produce ‘master stock solutions’.

The concentration of each master stock solution is different for each inhibitor, and is determined experimentally as (1) the concentration which will produce full inhibition of the cellular signaling pathway, and (2) will dissolve in ethanol.

Should the compound not dissolve in ethanol at the required master stock concentration, more ethanol is added until it is fully dissolved, and the dilutions (see below) are adjusted accordingly. For example: 8 mg of the compound SB203580 is dissolved in 2.12 mls ethanol to produce a master stock solution of 10 mM concentration.

Part of each master stock solution is further dissolved in ethanol to produce a diluted stock concentration. For example: a master stock solution of the compound SB203580 at 10 mM concentration is diluted 1:1 in ethanol to produce a 5 mM solution.

This process is repeated to produce 6 diluted solutions plus the master stock. The range of 6 dilutions is different for each inhibitor, and is determined experimentally as the range which will span the EC₅₀ and IC₅₀ for that inhibitor. For example: for the compound SB203580 there will be solutions of 10 mM (the master stock), 5 mM, 1 mM, 500 μM, 100 μM, 50 μM, 10 μM.

The solutions (6 dilutions plus master) are dispensed to 7 wells of one column of a 96-well polypropylene plate. The final well of the column of 8 wells is left empty to allow the customer to fill with it the solvent used to dissolve the inhibitor. This process is repeated for each inhibitor, until the 96-well plate is complete.

The plate is centrifuged under vacuum until the ethanol has evaporated. The inhibitor compounds are present in the base of the well as a thin layer or a solid plug, depending on the amount of each inhibitor.

The plate is sealed in the presence of an inert gas, such as argon.

### Possible Variations in Method of Production:

The inhibitor compounds can be dissolved in solvents other than ethanol. Possible organic solvents include ethanol, methanol, water, dimethyl formamide (DMF), and dimethyl sulfoxide (DMSO). The advantages of each using the criteria of solubility, volatility, toxicity, and manufacturing feasibility are shown in Table 1 below.

TABLE 1

| | | Volatility | | |
|---|---|---|---|---|
| | | (evaporation at | Toxicity | |
| | | temperatures at | (to humans, | |
| | Compound | which compounds | and in cell | Manufacturing |
| Solvent: | solubility | are stable) | culture) | feasibility |
| Ethanol | Acceptable (most | Excellent(boiling | Low risk | Excellent (standard |
| | compounds can be | point of 78° C., | | vacuum centrifuges |
| | dissolved at | lower under vacuum) | | can safely handle |
| | required | | | the vapour) |
| | concentration | | | |
| | for master stock | | | |
| | solution) | | | |
| Methanol | Acceptable | Excellent (boiling | Moderate | Excellent |
| | | point of 65° C., | risk | |
| | | lower under vacuum) | | |
| Water | Poor (few | Acceptable (boiling | Safe | Excellent |
| | compounds can be | point of 100° C., | | |
| | dissolved) | lower under vacuum) | | |
| DMF | Excellent(all | Poor (boiling point | High risk | Poor (special |
| | compounds can be | of 153° C.) | | centrifuge required, |
| | dissolved) | | | and not available |
| | | | | for testing) |
| DMSO | Excellent | Poor (boiling point | Moderate | Poor |
| | | of 189° C.) | risk | |

The dilutions and dispensing to plates could be performed by a robot. The production of the master stock and the method of dilution can be performed by any means that results in accurate concentrations of compound. The procedure should take the least time possible, as the solutions evaporate during the procedure.

Using these criteria, methanol is a possible evaporative alternative to ethanol. It is, however, not a widely used solvent in cell culture.

The 96-well plates (i.e. the array) should preferably be polystyrene rather than polypropylene. Polystyrene has less chemical resistance than polypropylene. This may not matter for storing the dry compound, but polystyrene would not be preferred as, once the compounds are re-suspended by the customer in DMSO, the lower chemical resistance could become an issue.

The 96-well plates could be replaced by separate strips of 8 wells, which could number 12 or fewer. Most 8-well strips are made of polystyrene. Several polypropylene strips of the form used for PCR have been tested, but these are optimized for heat transfer, and are thus thin-walled, and too flimsy for the purpose. If strips were used, a rack or similar container should be provided.

The plate can be centrifuged in the absence of a vacuum. This increases the time required for evaporation but, as discussed earlier, use of the centrifuge pushes the drug to the base of the well thus maximizing the accuracy of the reconstitution step. The amount of solvent the customer is required to dissolve the compound in can be a little as 35 μl. This will cover the base of the well. If compound is present too far up the walls, and is not dissolved on reconstitution, the concentration of the re-suspended solution can have reduced accuracy.

The plate can also be evaporated in the absence of centrifugation as solvents such as ethanol evaporate spontaneously. The use of a vacuum can be used to speed up the rate of evaporation. Evaporation without centrifuging can result in some of the inhibitor compound being present on the walls of the well in addition to the base of the well. As a result, this option, while possible, is less preferred than evaporation together with centrifuging. Use of a vacuum as well is most preferred.

The plate can be sealed in the presence of any inert gas (argon, nitrogen, or any other readily available gas). The intention is to exclude oxygen and water vapour from the plate environment.

The plate can be sealed with any sealing film or cap arrangement that excludes air, and forms a good seal over the lip of each well. Aluminium sealing foil deforms under light pressure (such as finger pressure) and thus forms a good seal, which can be complemented by a suitable adhesive. Adhesive is usually present on such sealing films for that reason. Most plastic films do not mould to the shape of the plate, and rely on adhesive. Caps for individual wells, that are connected in a strip of 8, can also be used. These take longer to apply (thus losing the argon gas). Caps for individual wells, that are connected in a mat of 96, can also be used. A cap seal of a suitable material could also be manufactured in a bespoke manner to fit and seal an array of wells.

The plate used for the array in all forms of the present invention can be packaged in any form that excludes air and light. Oxygen and water vapour may degrade the compounds. Some compounds may also be degraded by excessive light, so it is preferred that the foil pouch, or the array itself, excludes light. Packaging that includes temperature control aspects may also be preferred.

The plate and the additional components (plate sealing film, capstrips, product sheet) can be packaged in any form that protects the plate during shipping, although, as mentioned above, it is preferred that the packaging should exclude water and light.

The plate should also be protected from mechanical shock, which may cause the plug of inhibitor compound to dislodge from the base of the well. No adhesive or binding protein is used to stick the compound to the well following evaporation. Should the plug/fragment/particle of compound dislodge from the well, and the plate not remain upright for the rest of the journey, the compound may stick to the adhesive seal on the well, and therefore be lost when the seal is removed by the customer. If this happens, there is no compound to re-suspend, and also the customer may not notice it is missing.

### EXAMPLES

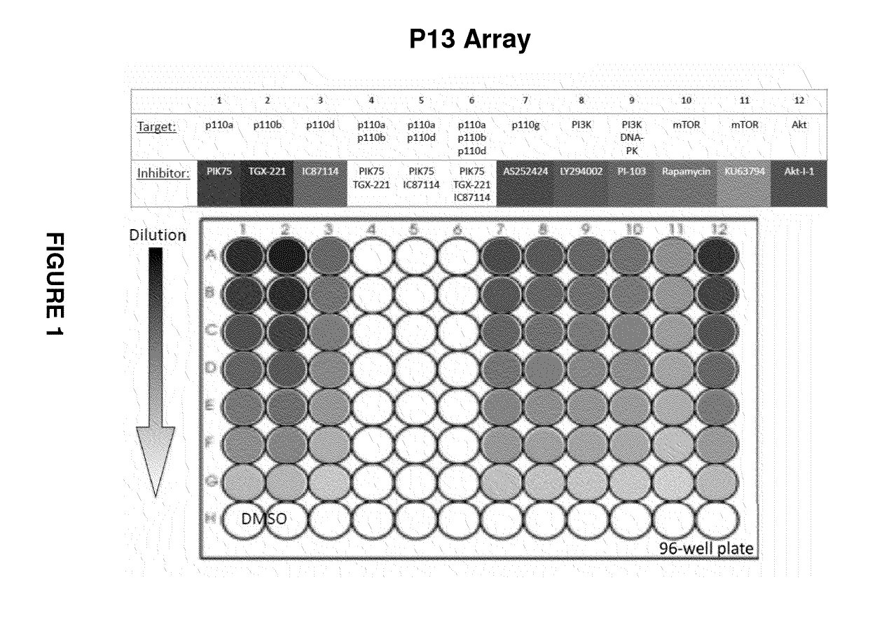

By way of example, and with reference to FIG. 1, an array is shown that targets the PI3K family.

The phosphatidylinositol 3-kinase (PI3K) family comprises eight enzymes, or isoforms. Some of these isoforms are important in insulin signalling, while others have been implicated in cancer, thrombosis, inflammation and cell survival. Therefore, it is very important to know which isoform is being activated by whatever stimulus, and to be able to inhibit some isoforms, while leaving others functional. To investigate isoform activation and also potential redundancy, the PI3K array contains isoform-specific inhibitors, as well as combinations of these isoform-specific inhibitors.

The format of the array is a 96-well microplate (1) containing 12 columns, each with one inhibitor, and 8 rows (indicated as A-H), each containing progressive dilutions of inhibitor, except for the final row (H) which contains DMSO as a negative control. If the array was to be prepared for transportation using evaporative methods, this final row (H) would be left empty, allowing its use later as a negative control.

Each well has sufficient solution for 5 assay points, or 5 ul, assuming that the solution is diluted 1/1000 by the user, and that 1 ml per assay point is required (for many applications, the amount required will be less than 1 ml). For the combinations of isoform-selective inhibitors, a separate master mix will be made up at the appropriate concentrations, so that the dilution procedure by the user is the same. Note that the use of combinations triples the total amount of isoform-selective inhibitor required per plate. The total amount of drug required for each column varies for each inhibitor and is shown in Table 2 below.

TABLE 2

| | 1 | 2 | 3 | 4 | 5 | 6 |
|---|---|---|---|---|---|---|
| Target: | p110a | p110b | p110d | p110a/b | p110a/d | p110a/b/d |
| Inhibitor: | PIK75 | TGX-221 | IC87114 | PIK75 | PIK75 | PIK75 |
| | | | | TGX-221 | IC87114 | TGX-221 |
| | | | | | | IC87-221 |
| | 7 | 8 | 9 | 10 | 11 | 12 |
| Target: | p110g | PI3K | PI3K/ | mTOR | mTOR | Akt/PKB |
| | | | DNA-PK | | | |
| | | | mTOR | | | |
| Inhibitor: | AS252424 | LY294002 | PI-103 | Rapamycin | KU0063794 | GSK690693 |

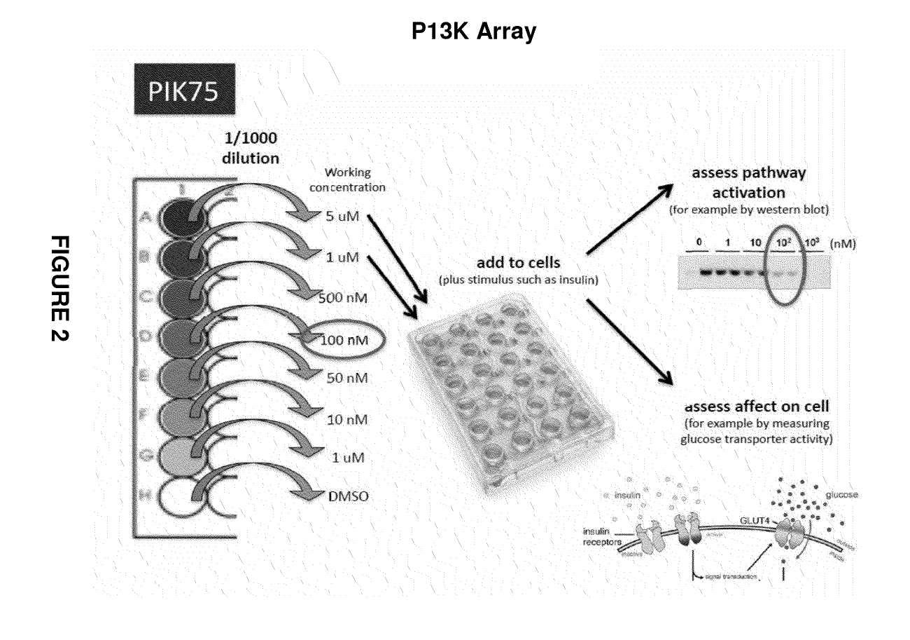

With general reference to FIG. 2, the dilutions for each inhibitor range from one-tenth of that inhibitor's IC₅₀ to one hundred times the IC₅₀. For most of the inhibitors, the most concentrated inhibitor solution on the plate is 5 mM in DMSO. As seen in FIG. 2, the inhibitor drug from the micro-assay plate of FIG. 1 is added to the target cells (optionally together with a suitable stimulus compound, such as insulin). This then allows analysis of effect via options such as western blot analysis to assess pathway activation, and/or to assess the effect on the cells themselves by measuring activity such as glucose transporter activity for example.

Dilutions of the inhibitor drug on the assay plate is shown in Table 3:

TABLE 3

| | 1 | | 2 | | 3 | | 4 | 5 | 6 |
|---|---|---|---|---|---|---|---|---|---|
| Inhibitor: | PIK75 | | TGX-221 | | PIK294 | | PIK75 | PIK75 | PIK75 |
| | | | | | | | TGX-221 | PIK294 | TGX-221 |
| | | | | | | | | | PIK294 |
| Dilution*: | 1 | mM | 2.5 | mM | 1 | mM | (as for single inhibitor) | (as for | (as for |
| | | | | | | | | single | single |
| | | | | | | | | inhibitor) | inhibitor) |
| | 500 | uM | 1 | mM | 500 | μM | (as for single inhibitor) | (as for | (as for |
| | | | | | | | | single | single |
| | | | | | | | | inhibitor) | inhibitor) |
| | 100 | uM | 500 | uM | 100 | μM | (as for single inhibitor) | (as for | (as for |
| | | | | | | | | single | single |
| | | | | | | | | inhibitor) | inhibitor) |
| | 50 | uM | 100 | uM | 50 | uM | (as for single inhibitor) | (as for | (as for |
| | | | | | | | | single | single |
| | | | | | | | | inhibitor) | inhibitor) |
| | 10 | uM | 50 | uM | 10 | uM | (as for single inhibitor) | (as for | (as for |
| | | | | | | | | single | single |
| | | | | | | | | inhibitor) | inhibitor) |
| | 5 | uM | 10 | uM | 5 | uM | (as for single inhibitor) | (as for | (as for |
| | | | | | | | | single | single |
| | | | | | | | | inhibitor) | inhibitor) |
| | 1 | uM | 5 | uM | 1 | uM | (as for single inhibitor) | (as for | (as for |
| | | | | | | | | single | single |
| | | | | | | | | inhibitor) | inhibitor) |

| | | | | | |
|---|---|---|---|---|---|
| DMSO | DMSO | DMSO | (as for single inhibitor) | (as for | (as for |
| | | | | single | single |
| | | | | inhibitor) | inhibitor) |

| | 7 | | 8 | | 9 | | 10 | | 11 | | 12 | |
|---|---|---|---|---|---|---|---|---|---|---|---|---|
| Inhibitor: | AS252424 | | LY294002 | | PI-103 | | Rapamycin | | KU0063794 | | GSK690693 | |
| Dilution*: | 10 | mM | 50 | mM | 1 | mM | 5 | mM | 1 | mM | 5 | mM |
| | 5 | mM | 10 | mM | 500 | uM | 1 | mM | 500 | uM | 1 | mM |
| | 1 | mM | 5 | mM | 100 | uM | 500 | uM | 100 | uM | 500 | uM |
| | 500 | uM | 1 | mM | 50 | uM | 100 | uM | 50 | uM | 100 | uM |
| | 100 | uM | 500 | uM | 10 | uM | 50 | uM | 10 | uM | 50 | uM |
| | 50 | uM | 100 | uM | 5 | uM | 10 | uM | 5 | uM | 10 | uM |
| | 10 | uM | 50 | uM | 1 | uM | 5 | uM | 1 | uM | 5 | uM |

| | | | | | |
|---|---|---|---|---|---|
| DMSO | DMSO | DMSO | DMSO | DMSO | DMSO |

*The dilutions highlighted in red are those that when diluted 1/1000 by the user, give approximately the IC₅₀ for that inhibitor.

If the array was to be prepared for transportation using evaporative methods, this final row showing the presence of DMSO only would be left empty, allowing its use later as a negative control.

The amount of inhibitor drug required per assay plate is shown in Table 4:

TABLE 4

| | 1 | 2 | 3 | 4 | 5 | 6 |
|---|---|---|---|---|---|---|
| Inhibitor: | PIK75 | TGX-221 | IC87114 | PIK75 | PIK75 | PIK75 |
| | | | | TGX-221 | IC87114 | TGX-221 |
| | | | | | | IC87-221 |

| | | | | |
|---|---|---|---|---|
| Amount (ug) | 60 | 36 | 99 | (included in total for single) |

| | 7 | 8 | 9 | 10 | 11 | 12 |
|---|---|---|---|---|---|---|
| Inhibitor: | AS252424 | LY294002 | PI-103 | Rapamycin | KU0063794 | GSK690693 |
| Amount (ug) | 25 | 115 | 12 | 30 | 16 | 14 |

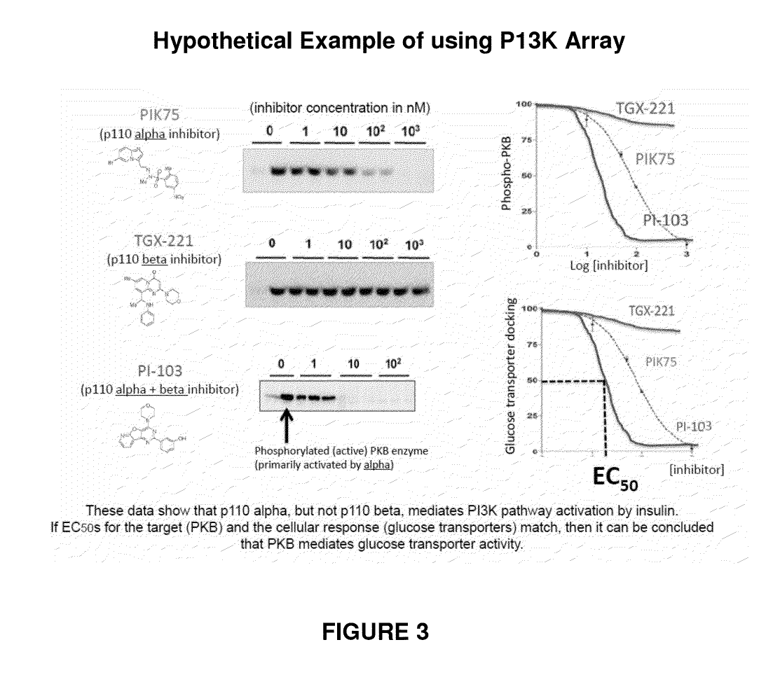

With general reference to FIG. 3, a hypothetical example of the use of a PI3K micro-array as shown in FIG. 1 is provided. The analysis provided via western blot analysis of pathway activation and also of cellular response allows a determination to be made of the effect of a target (PKB) on glucose transporter activity.

To transport the array referred to in FIG. 1 ready made in DMSO, the array would preferably be frozen, purged with an inert gas, and then the wells sealed. Reconstitution for use would be by defrosting processes.

In order to transport the array referred to in FIG. 1 using evaporative methods, it would be preferred to dissolve the inhibitor drugs in ethanol, then remove the ethanol from the solution by centrifuging under vacuum. The wells holding the drugs in the array would be purged with an inert gas and then the wells sealed. Reconstitution of the drug with the same amount of DMSO diluent would be completed following transport for use.

### Example 2

### I. Production Process of Batch 001 of MAPK Easy50 Array #IAM001

Inhibitor compounds in powder form were dissolved in ethanol (analytical grade, Merck #100983.1000) to produce ‘master stock solutions’ of the following concentrations:
SB590885: 10 mg dissolved to 1.25 mM
Sorafenib: 54 mg dissolved to 6.25 mM
CI1040: 1.5 mg dissolved to 1 mM
PD325901: 1.65 mg dissolved to 1 mM
AZD6244: 1.5 mg dissolved to 0.25 mM
PD98059: 7 mg dissolved to 2.5 mM
BIRB796: 5 mg dissolved to 2.5 mM
SB203580: 8 mg dissolved to 10 mM
RWJ67657: 4 mg dissolved to 5 mM
SB202190: 7 mg dissolved to 10 mM
Tak715: 9 mg dissolved to 10 mM
R1487: 4 mg dissolved to 5 mM

Solutions were made in 15 ml tubes and stored at room temperature in the dark overnight. The tubes containing dissolved compound were shaken and briefly centrifuged (Eppendorf 5810R).

Part of each master stock solution was further dissolved in ethanol to produce diluted stock concentrations as shown in the Table 5 below. Solutions were made up in 1.5 ml tubes.

TABLE 5

| | Col 1 | Col 2 | Col 3 | Col 4 | Col 5 | Col 6 |
|---|---|---|---|---|---|---|
| Compound | SB590885 | Sorafenib | CI1040 | PD325901 | AZD6244 | PD98059 |
| Master stock | 1.25 | 6.25 | 1 | 1 | 0.25 | 2.5 |
| conc. (mM) | | | | | | |
| Dilution of master | | | | | | |
| stock intotal | | | | | | |
| volume | | | | | | |
| Row A | | | undiluted | undiluted | | |
| Row B | | | 1/2 | 1/2 | | |
| Row C | 1/1.25 | | 1/10 | 1/10 | 1/2.5 | 1/2.5 |
| Row D | 1/2.5 | 1/1.25 | 1/20 | 1/20 | 1/5 | 1/5 |
| Row E | 1/12.5 | 1/6.25 | 1/100 | 1/100 | 1/25 | 1/25 |
| Row F | 1/25 | 1/12.5 | 1/200 | 1/200 | 1/50 | 1/50 |
| Row G | 1/125 | 1/62.5 | 1/1000 | 1/1000 | 1/250 | 1/250 |

| | Col 7 | Col 8 | Col 9 | Col 10 | Col 11 | Col 12 |
|---|---|---|---|---|---|---|
| Compound | BIRB | SB203580 | RWJ | SB202190 | Tak715 | R1487 |
| Master stock | 2.5 | 10 | 5 | 10 | 10 | 5 |
| conc. (mM) | | | | | | |
| Dilution of master | | | | | | |
| stock intotal | | | | | | |
| volume | | | | | | |
| Row A | | undiluted | undiluted | undiluted | undiluted | undiluted |
| Row B | 1/2.5 | 1/2 | 1/5 | 1/2 | 1/2 | 1/5 |
| Row C | 1/5 | 1/10 | 1/10 | 1/10 | 1/10 | 1/10 |
| Row D | 1/25 | 1/20 | 1/50 | 1/20 | 1/20 | 1/50 |
| Row E | 1/50 | 1/100 | 1/100 | 1/100 | 1/100 | 1/100 |
| Row F | 1/250 | 1/200 | 1/500 | 1/200 | 1/200 | 1/500 |
| Row G | 1/500 | 1/1000 | 1/1000 | 1/1000 | 1/1000 | 1/1000 |

These solutions were transferred to a deepwell 96-well plate (2 ml capacity per well, Eppendorf #0030522.109), the plate being orientated such that the row and column labels on the plate matched those in the table above.

Six polypropylene 96-well plates (Matrix #4919) were orientated such that the row and column labels on the plates matched those in the deepwell plate.

Using a multichannel pipette set to 36 μl, the diluted solutions from Column 1 of the deepwell plate were transferred to Column 1 of each of the six 96-well plates.

This process was repeated for columns 2-12, using new tips for each column.

The deepwell plate was then sealed with sealing film (Sigma #A5596) to minimize evaporation.

Then solutions were dispensed to the ‘special wells’—those wells requiring non-standard volumes of master stock solution, as shown in the Table 6 below. Amounts of master stock were dispensed using a single-channel pipette directly to the appropriate wells of the six polypropylene 96-well plates. New tips were used for each solution.

TABLE 6

| | Col 1 | Col 2 | Col 5 | Col 6 | Col 7 |
|---|---|---|---|---|---|
| | SB590885 | Sorafenib | AZD6244 | PD98059 | BIRB |
| Volumes (μl): | 140 | 140 | 140 | 140 | 70 |
| Row A | | | | | |
| Volumes (μl): | 140 | 140 | 70 | 70 | |
| Row B | | | | | |
| Volumes (μl): | | 56 | | | |
| Row C | | | | | |

Row H of each polypropylene 96-well plate was left empty.

The six plates were carefully placed in the rotor of a vacuum centrifuge (Savant SpeedVac Concentrator #SPD111V). The centrifuge was set to 35° C. and run at the fixed speed and vacuum level of the unit.

The plates were centrifuged until the ethanol had visibly evaporated and the inhibitor compounds were present in the base of the well as a thin layer or a solid plug, depending on the amount of each inhibitor (approximately 40 minutes).

Then further solution was dispensed to certain ‘special wells’ as shown in the Table 7 below.

This was because the capacity of the well is less than the total amount of solution required for that well.

TABLE 7

| | Col 1 | Col 2 |
|---|---|---|
| | SB590885 | Sorafenib |
| Volumes (μl): | 140 | 140 |
| Row A | | |

To balance the plate during centrifugation, two wells on the opposite side of the plate, wells 11A and 12A, had the same volume (140 μl) of pure ethanol pipetted into them.

The plates were centrifuged until the ethanol had visibly evaporated (approximately 20 minutes).

The plates were placed in a large plastic box, adjacent to a small tank of argon (D-size tank, Instrument-grade argon) which was fitted with a regulator and hose-pipe. The argon flow rate was set low, and adjusted so that the pressure of the gas on a fingertip was steady but not forceful. The end of the hose was placed just inside the box, with the plastic nozzle on the end of the pipe directed so that the jet of gas did not displace powdered compound at the base of the wells.

The box was flushed with argon for two minutes, during which a lid loosely covered the box but was not sealed. Then the hose was removed, the lid sealed, and it was left for 10 minutes.

The box was opened and aluminium sealing film (Corning #6570) was quickly and firmly applied to each of the plates.

The plates were then sealed inside foil pouches (Cellofresh SU3 SSM) along with a dessicant sachet (Sigma #Z163562).

Note that many of the steps were done concurrently—i.e. while one set of six plates was in the vacuum centrifuge, solutions were being dispensed to another set of six plates. The batch was produced within one day.

### II. Testing Process of Batch 001 of MAPK Easy50 Array #IAM001

### Method:

Hek293 cells at passage 17 were grown to confluence in six 12-well plates with 1 ml of medium (DMEM with antibiotics and 10% fetal bovine serum) per well. One plate of cells was used to test one column of the array plate (i.e. one inhibitor).

One plate from batch001 was chosen at random and unsealed. Using a multichannel pipette, 35 μl of dimethyl sulfoxide (DMSO, Sigma #D2650) was dispensed to each well of the plate, using new tips for each column.

The plate was sealed with sealing film (Sigma #A5596), and placed on a platform rocker for 30 minutes to dissolve the compounds.

In a cell culture hood, the plate was unsealed. 1 μl from Column 1, Row A was dispensed to a cell culture well. This was repeated for Rows B through G. For the two cell culture wells that would not be exposed to inhibitor compound (untreated; stimulus without inhibitor), 1 μl of pure DMSO from row H of the plate was used as the negative control.

This was repeated with another plate of cells for Column 2. The plates were slowly rotated so as to gently disperse the inhibitor solutions through the cell culture medium.

The cells were returned to the incubator for 10 minutes.

The cells were then exposed to the stimulus for the MAPK signaling pathway: Hepatocyte Growth Factor (Symansis #6003C) at 20 ng/ml for 5 minutes (except for the untreated control cells).

The cells were washed in PBS then stored at −80° C.

This process was repeated for Columns 3 and 4 of the plate, then Columns 5 and 6.

The plates were thawed on ice and the cells lysed in 100 μl Lysis Buffer (Symansis #CLB001) per well. The protein content was measured by BCA assay (Pierce #23227).

Cell samples were tested for inhibition of MAPK signaling pathway using ELISA for phospho-ERK (Symansis #MKA005).

The ELISA plates were set up as shown in Table 8 below.

TABLE 8

| | | | | | | | | |
|---|---|---|---|---|---|---|---|---|
| PLATE1 | 1 | 2 | 3 | 4 | 5 | 6 | 9 | 10 |

| | | | | |
|---|---|---|---|---|
| A | blank | 1B | 2C | 4HGF |
| B | 1un | 1A | 2B | 4G |
| C | 1HGF | 2un | 2A | 4F |
| D | 1G | 2HGF | blank | 4E |
| E | 1F | 2G | blank | 4D |
| F | 1E | 2F | 3un | 4C |
| G | 1D | 2E | 3HGF | 4B |
| H | 1C | 2D | 3G | 4A |

| | | | | | | |
|---|---|---|---|---|---|---|
| PLATE2 | 1 | 2 | 3 | 4 | 5 | 6 |

| | | | |
|---|---|---|---|
| A | blank | 5B | 6D |
| B | 5un | 5A | 6C |
| C | 5HGF | blank | 6B |
| D | 5G | 6un | 6A |
| E | 5F | 6HGF | blank |
| F | 5E | 6G | Hek 30.9.9 un |
| G | 5D | 6F | Hek 30.9.9 |
| | | | stim |
| H | 5C | 6E | blank |

20 μg of each cell sample was diluted in assay buffer, and each sample was tested in duplicate. Assay buffer was used as a blank. Cell samples were incubated in the ELISA wells for 2 hours at room temperature, on a rocking platform.

Wells were washed, then detection antibody added for 2 hours at room temperature, on a rocking platform.

Wells were washed, then sHRP added for 30 minutes at room temperature, on a rocking platform.

Wells were washed, then TMB added for 20 minutes at room temperature, in the dark.

The reaction was stopped and absorbances read at 450 nm. Raw absorbance values were corrected by subtracting a blank value.

### Results:

In previous experiments (as described in Example 1 above) the powdered compounds were first dissolved in DMSO, and applied to cells at the same final concentrations, and in the same manner as described above. The cells were lysed and the MAPK pathway stimulation was measured by phospho-ERK ELISA in the same manner. This process was repeated with the inhibitor drugs CI1040, PD98059, and PD325901 and compared to the results of the use of those inhibitor drugs following ethanol dissolution and evaporation as set out in Example 2.

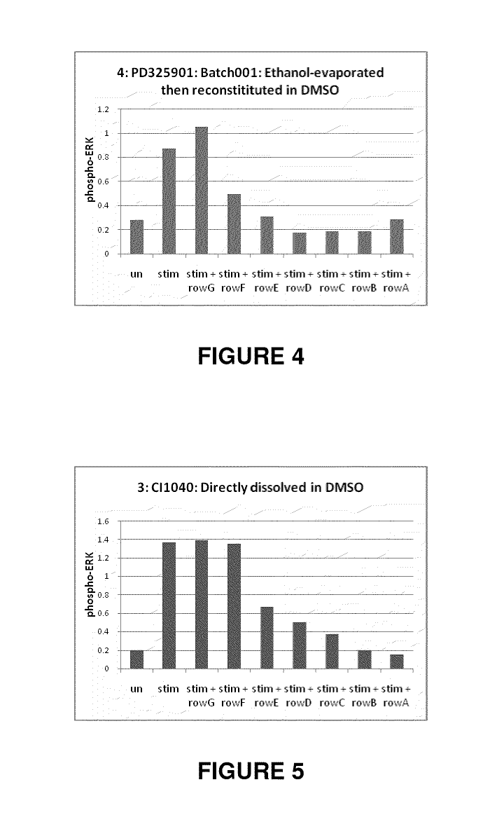

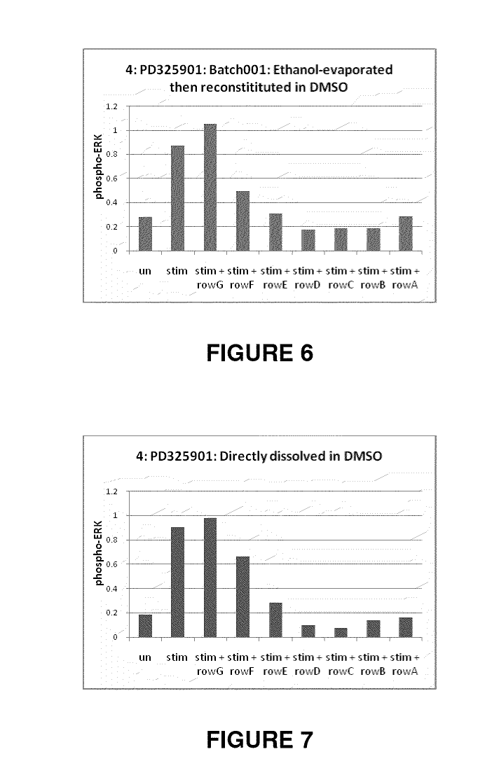

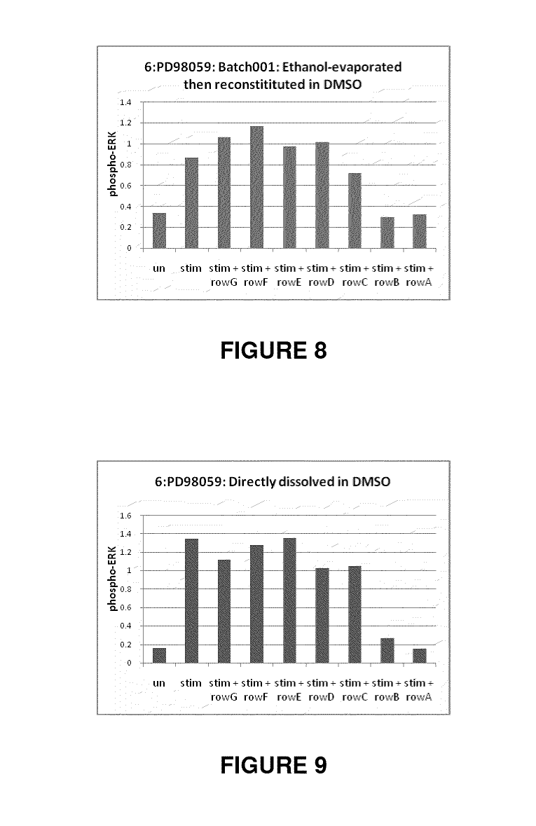

The results were plotted and compared with previous data. The results are shown in FIGS. 4 to 9.

FIGS. 4 to 9 show that the amount of phosphorylated ERK decreased with increasing concentration of inhibitor compound, regardless of whether the inhibitor was directly dissolved in DMSO, or first dissolved in ethanol, then evaporated, and finally re-constituted in DMSO. The degree of inhibition is similar in both cases.

### CONCLUSION

There is no significant effect of the ethanol-evaporation procedure on the effectiveness of the inhibitors on cells in vitro.

The present invention allows accurate determination of issues such as those indicated by the Examples and Figures by research teams for reasonable cost outlay, as they will not need to obtain the drugs and prepare the arrays themselves. This is particularly as a result of the inventors recognition of the ability to use evaporative techniques to create transportable and reconstitutable arrays. As a result, more research will be able to undertaken by more research teams into important physiological issues thus increasing the likelihood of beneficial results. In addition the invention also allows for the transportation and accurate reconstitution of multiplicities of pharmaceutical drug containing compositions. As a result, provided that the carrier (preferably a solvent) for reconstituting the pharmaceutical composition is available, the composition can be formulated and used at a place and time desired by the user.

The foregoing describes the invention including preferred forms thereof. Alterations and modifications as would be readily apparent to a person skilled in this art are intended to be included within the scope of the invention disclosed and claimed in the attached claims.

1. A method of forming a plurality of re-constitutable doses of at least one drug in a plurality of wells, the method including the steps of (i) placing a known amount of said drug in a suitable carrier to form a first composition having a known concentration (ii) placing at least two selected amounts of that first composition into individual wells and (iii) converting the first composition into a transportable form that can later be converted into a second composition having a known concentration and (iv) sealing the wells.

2. The method according to claim 1 wherein the plurality of re-constitutable doses of at least one drug is an array of a plurality of drugs that target cell signalling molecules, and steps (i) and (ii) include determining a series of dilutions for each of the selected drugs that span the EC50 of the molecular target of the selected drugs and dispensing an amount of each of the selected drugs into a series of wells such that when a fixed amount of the selected drugs is transferred from each well to a series of fixed volumes of the molecular target, the final range of concentrations created spans the EC50 of the molecular target (determined previously), and step (iii) includes purging the series of wells with a suitable gas prior to sealing the wells.

3. The method according to claim 1 wherein the plurality of re-constitutable doses of at least one drug is an array of at least one drug and step (i) includes determining the desired concentration(s) of the selected drug(s) and forming a first composition of the selected drug(s) having that desired concentration.

4. The method according to claim 1 wherein the first composition is converted into a transportable form by:
A) freezing the first composition wherein the first composition and the second composition are the same;
B) evaporating the suitable carrier from the first composition;
C) centrifuging and evaporating the suitable carrier from the first composition; or
D) freeze drying the first composition.

5-8. (canceled)

9. The method according to claim 4 wherein the transportable form is a powder or crystalline form of the drug.

10. The method according to claim 1 wherein the drug is selected from kinase inhibitors, antibiotics, pain relief drugs, anti-inflammatory drugs, trauma medication, and psychiatric drugs, and the suitable carrier is a solvent selected from ethanol, methanol, water, DMF or DMSO.

11-12. (canceled)

13. The method according to claim 1 wherein the first and the second compositions are solutions, suspensions, dispersions or emulsions.

14. The method according to claim 1 wherein the wells are purged with an inert gas, more preferably nitrogen, argon or the like, prior to sealing.

15. The method according to claim 1 wherein the seals allow opening and re-sealing of individual or a plurality of wells and are optionally capable of at least minimising evaporation and cross-contamination.

16-17. (canceled)

18. The method according to claim 1 wherein step (iii) includes evaporating the suitable carrier from the first composition such that the drugs are converted into a powder or crystalline form and remain in their individual wells, and are later converted into the second composition using the same or a different carrier as used in the first composition.

19. (canceled)

20. A method of providing an array of drugs that target cell signalling molecules, the method including the steps of:
A) selecting a plurality of drugs each of which targets a cell signalling molecule;
B) determining a series of dilutions for each of the selected drugs that span the EC₅₀ of the molecular target of the selected drugs;
C) dispensing an amount of each of the selected drugs into a series of wells;
D) purging the wells with a suitable gas;
E) sealing the wells; and
wherein, the amount of each of the selected drugs dispensed in the wells is such that, when a fixed amount of the selected drugs is transferred from each well to a series of fixed volumes of the molecular target, the final range of concentrations created spans the EC₅₀ of the molecular target (determined in B) and optionally also the IC₅₀ of the molecular target of the selected drugs.

21. A method as claimed in claim 20 wherein the method further includes the further step of transforming the amount of each of the selected drugs in the wells into a transportable form capable of reconstitution after step C) and before step D), wherein the amount of each of the selected drugs dispensed in the wells is such that, when the selected drugs are reconstituted, a fixed amount of the selected drugs is transferred from each well to a series of fixed volumes of the molecular target, the final range of concentrations created spans the EC₅₀ of the molecular target (determined in B) and optionally also the IC₅₀ of the molecular target of the selected drugs.

22. The method according to claim 20 wherein the molecular target is an appropriate cell or tissue culture media.

23. The method according to claim 20 wherein the drug is dispensed into the well in a solution of suitable diluent.

24. (canceled)

25. The method according to claim 23, wherein the diluent is removed by evaporation to leave the drug in a transportable form in the wells such that when a fixed amount of diluent is later added to the wells, a series of concentrations of the drug is created for transfer to the fixed volumes of the molecular target.

26-27. (canceled)

28. The method according to any claim 20 wherein the method includes reconstitution by appropriate re-suspension or dissolution of the selected drug in the well and subsequent dilution of the selected drug in the cell/tissue culture media.

29. (canceled)

30. The method according to claim 20 wherein the selected drug is selected from kinase inhibitors, more preferably PI3 kinase and MAP kinase inhibitors.

31. The method according to claim 20 wherein the wells are purged with an inert gas, more preferably nitrogen, argon or the like.

32. The method according to claim 20 wherein the dilutions span 1/10th of the selected drug's IC₅₀ to 100× that drug's IC₅₀.

33. The method according to claim 20 wherein the array is used to determine metabolic pathway activation or cell signalling pathway activation.

34-43. (canceled)

44. The method according to claim 20 wherein the series of wells is contained in a multiwall plate containing a plurality of series of different drugs thus allowing the creation of an array that covers the EC50, and optionally the IC50, of a plurality of different cell signalling molecules.

45-49. (canceled)

50. An array including a plurality of well strips, each well strip containing a selected drug which targets a cell signalling molecule, each well strip including a plurality of wells containing a either a sequence of dilutions of the selected drug that span the EC₅₀, and optionally the IC₅₀, of the molecular target of the drug, or the selected drug in a form capable of dilution to create a sequence of dilutions of the selected drug that span the EC₅₀, and optionally the IC₅₀, of the molecular target of the drug, each well also including an inert gaseous environment and being sealed to contain the selected drugs in that inert environment.

51-52. (canceled)

53. The array according to claim 50 wherein the form of the selected drug capable of dilution to create a sequence of dilutions of the selected drug that span the EC₅₀ and preferably the IC₅₀ of the molecular target of the drug, is a powder or crystalline form.

54-55. (canceled)

56. The array according to claim 50 wherein the seals allow opening and re-sealing of individual or a plurality of wells and are optionally capable of resealing the wells following puncture access (e.g., by syringe needle) to the diluted drugs therein.

57. (canceled)

58. The array according to claim 50 wherein the series of dilutions span 1/10th of the drug's IC₅₀ and EC₅₀ to 100× that drug's IC₅₀ and EC₅₀.

59. The array according to claim 50 wherein the array also includes wells including the only the diluent as a negative control for each of the sequential drug dilutions.

60-64. (canceled)
